(19) United States
(12) Patent Application Publication
(10) Pub. No.: US 2004/0162428 A1
(43) Pub. Date: Aug. 19, 2004

(54) 6-(2,6-difluorophenyl)-triazolopyrimidines as fungicides

(76) Inventors: Jordi Tormo i Blasco, Limburgerhof (DE); Hubert Sauter, Mannheim (DE); Bernd Muller, Frankenthal (DE); Markus Gewehr, Kastellaun (DE); Wassilios Grammenos, Ludwigshafen (DE); Thomas Grote, Wachenheim (DE); Andrea Gypser, Mannheim (DE); Joachim Rheinheimer, Ludwigshafen (DE); Ingo Rose, Mannheim (DE); Peter Schafer, Ottershiem (DE); Frank Schieweck, Hessheim (DE); Eberhard Ammermann, Heppenheim (DE); Siegfried Strathmann, Limburgerhof (DE); Gisela Lorenz, Hambach (DE); Reinhard Stierl, Muttterstadt (DE)

Correspondence Address: KEIL & WEINKAUF, 1350 CONNECTICUT AVENUE, N.W., WASHINGTON, DC, 20036, US

(21) Appl. No.: 10/483,597

(22) PCT Filed: Jul. 8, 2002
(86) PCT No.: PCT/EP02/07575

(30) Foreign Application Priority Data
Jul. 18, 2001 (EP) 01117404.2

(51) Int. Cl. C07D 487/04

(52) U.S. Cl. 544/60; 544/118; 544/263

(57) ABSTRACT

6-(2,6-Difluoro-phenyl)-triazolopyrimidines of formula (I), in which R¹ and R² independently denote hydrogen or Alkyl, Alkyl, Alkenyl, Alkynyl, or alkadienyl, cycloalkyl, phenyl, naphthyl, or 5- or 6-membered heterocyclyl, containing one to four nitrogen atoms or one to three nitrogen atoms and one sulphur or oxygen atom. Or 5- or 6-membered heteroaryl, containing one to four nitrogen atoms or one to three nitrogen atoms and one sulphur or xygen atom where R¹ and R² radicals may be unsubstituted or substituted as defined in the description, or R¹ and R² together with the interjacent nitrogen atom represent a 5- or 6-membered hetericyclic ring, containing one to four nitrogen atoms or one to three nitrogen atoms and one sulphur or oxygen atom, which may be substituted; X is halogen, cyano, alkoxy, haloalkoxy or alkenyloxy; Processes for their preparation, compositions containing them and to their use for combating phytopathogenic fungi.

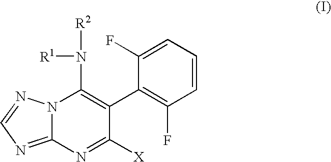

The invention relates to 6-(2,6-difluoro-phenyl)-triazolopyrimidines of formula I

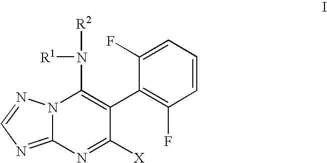

in which

R¹ and R² independently denote hydrogen or

C₁-C₁₀-alkyl, C₂-C₁₀-alkenyl, C₂-C₁₀-alkynyl, or C₄-C₁₀-alkadienyl, C₃-C₁₀-cycloalkyl, phenyl, naphthyl, or

5- or 6-membered heterocyclyl, containing one to four nitrogen atoms or one to three nitrogen atoms and one sulfur or oxygen atom, or

5- or 6-membered heteroaryl, containing one to four nitrogen atoms or one to three nitrogen atoms and one sulfur or oxygen atom, or

where R¹ and R² radicals may be unsubstituted or may carry one to three groups R^{a},

R^{a} is cyano, nitro, hydroxyl, C₁-C₆-alkyl, C₃-C₆-cycloalkyl, C₁-C₆-alkoxy, C₁-C₆-alkylthio, C₁-C₆-alkylamino, di-C₁-C₆-alkylamino, C₂-C₆-alkenyl, C₂-C₆-alkenyloxy, C₂-C₆-alkynyl, C₃-C₆-alkynyloxy and C₁-C₄-alkylenedioxy; or

R¹ and R² together with the interjacent nitrogen atom represent a 5- or 6-membered heterocyclic ring, containing one to four nitrogen atoms or one to three nitrogen atoms and one sulfur or oxygen atom, which may be substituted by one to three R^{a} radicals;

X is halogen, cyano, C₁-C₆-alkoxy, C₁-C₆-haloalkoxy or C₃-C₈-alkenyloxy.

Moreover, the invention relates to processes for their preparation, compositions containing them and to their use for combating phytopathogenic fungi.

6-Phenyl-7-amino-triazolopyrimidines are generally known from U.S. Pat. No. 4,567,262 and U.S. Pat. No. 5,593,996.

Triazolopyrimidines with a trifluorophenyl group in 6-position are disclosed in WO-A 98/46607 and EP-A 945 453.

From WO-A 98/46608 some 6-(2,6-difluoro-phenyl)-triazolopyrimidines are known, which are substituted in the 7-position by fluorinated alkylamines.

The compounds disclosed in the documents discussed above are said to be active against various phytopathogenic fungi.

It is an object of the present invention to provide compounds having improved fungicidal activity.

We have found that this object is achieved by the compounds defined at the outset. Furthermore, we have found processes for their preparation, compositions comprising them and methods for controlling phytopathogenic fungi using the compounds I.

The compounds of the formula I differ from the compounds known from WO-A 98/46608 in the combination of the 2,6-difluoro-phenyl group with an halogen free amino group in the 7-position of the triazolopyrimidine system.

The present invention further provides a process for the preparation of compound of formula I as defined above which comprises treating a 5,7-dihalo compound of formula II in which X is halogen with an amine of formula III.

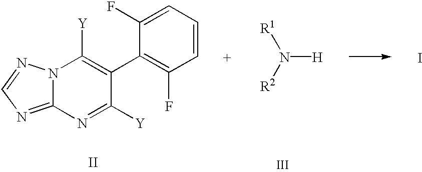

The reaction between the 5,7-dihalo compound II and the amine of formula III can be carried out under conditions known from WO-A 98/46608.

Compounds of formula II are known from EP-A 550 113; they can be prepared by known methods [cf. EP-A 550 113 or EP-A 770 615].

The reaction is preferably carried out in the presence of a solvent. Suitable solvents include ethers, such as dioxane, diethyl ether and, especially, tetrahydrofuran, halogenated hydrocarbons such as dichloromethane and aromatic hydrocarbons, for example toluene.,

The reaction is suitably carried out at a temperature in the range from 0° C. to 70° C., the preferred reaction temperature being from 10° C. to 35° C.

It is also preferred that the reaction is carried out in the presence of a base. Suitable bases include tertiary amines, such as triethylamine, and inorganic bases, such as potassium carbonate or sodium carbonate. Alternatively, an excess of the compound of formula III may serve as a base.

The reaction mixtures are worked up in a customary manner, for example by mixing with water, phase separation and, if required, chromatographic purification of the crude products. Some of the end products are obtained in the form of colorless or slightly brownish, viscous oils, which are purified or freed from volatile components under reduced pressure and at moderately elevated temperatures. If the end products are obtained as solids, purification can also be carried out by recrystallization or digestion.

Compounds of formula II are known in the art and can be obtained by synthesis routes disclosed in EP-A 550 113, EP-A 770 615 and WO-A 98/46608.

Compounds of formula I in which X denotes cyano, C₁-C₆-alkoxy, C₁-C₆-haloalkoxy or C₃-C₈alkenyloxy can be prepared by reating compounds I in which X is halogen, preferably chloro, with compounds of formula IV, which are, dependent from the value of X′ to be introduced to yield formula I compounds, an anorganic cyano salt, an alkoxylate, haloalkoxylate or an alkenyloxylate, respectively, preferably in the presence of a a solvent. The cation M in formula IV has minor influence; for practical and economical reasons usually ammonium-, tetraalkylammonium- or alkalimetal- and earth metal salts are preferred.

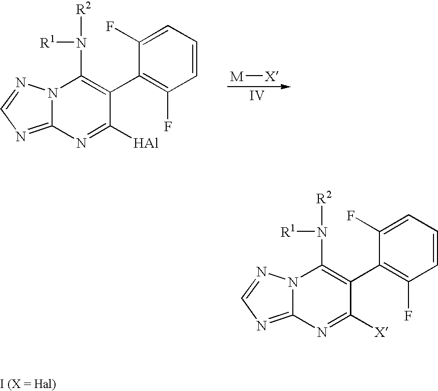

The reaction is suitably carried out at a temperature in the range from 0 to 120° C., the preferred reaction temperature being from 10 to 40° C. [cf. J. Heterocycl. Chem., Vol. 12, p. 861-863 (1975)].

Suitable solvents include ethers, such as dioxane, diethyl ether and, especially, tetrahydrofuran, halogenated hydrocarbons such as dichloromethane and aromatic hydrocarbons, for example toluene.

If individual compounds I are not obtainable by the routes described above, they can be prepared by derivatization of other compounds I.

In the symbol definitions given in the formulae above, collective terms were used which generally represent the following substituents:

halogen: fluorine, chlorine, bromine and iodine;

C₁-C₁₀-alkyl: saturated, straight-chain or branched hydrocarbon radicals having 1 to 10, especially 1 to 6 carbon atoms, for example C₁-C₄-alkyl as mentioned above or pentyl, 1-methylbutyl, 2-methylbutyl, 3-methylbutyl, 2,2-di-methylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, -1,1,2-trimethylpropyl, 1,2,2-trimethylpropyl, 1-ethyl-1-methylpropyl and 1-ethyl-2-methylpropyl;

C₂-C₁₀-alkenyl: unsaturated, straight-chain or branched hydrocarbon radicals having 2 to 10, especially 2 to 6 carbon atoms and a double bond in any position, for example ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl and 2-methyl-2-propenyl;

C₂-C₁₀-alkynyl: straight-chain or branched hydrocarbon radicals having 2 to 10, especially 2 to 4 carbon atoms and a triple bond in any position, for example ethynyl, 1-propynyl, 2-propynyl, 1-butynyl, 2-butynyl, 3-butynyl and 1-methyl-2-propynyl;

haloalkyl moieties of C₁-C₆-haloalkoxy: straight-chain or branched alkyl groups having 1 to 6, preferably 1 to 4 carbon atoms (as mentioned above), where the hydrogen atoms in these groups may be partially or fully replaced by halogen atoms as mentioned above, for example C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, fluoroethoxy, 2-chloro-2-fluoroethoxy, 2-chloro-2,2-difluoroethoxy, 2,2-dichloro-2-fluoroethoxy, 2,2,2-trichloroethoxy and pentafluoroethoxy;

C₃-C₁₀-cycloalkyl: mono- or bicyclic cycloalkyl groups having 3 to 10 carbon atoms; monocyclic groups preferably have 3 to 8, especially 3 to 6 ring members, bicyclic groups preferably have 8 to ring members.

A 5- or 6-membered heterocyclyl group, containing one to four nitrogen atoms or one to three nitrogen atoms and one sulfur or oxygen atom, preferably one oxygen atom.

5-membered heteroaryl, containing one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;

6-membered heteroaryl, containing one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

With respect to their intended use, preference is given to triazolopyrimidines of the formula I having the following substituents, where the preference is valid in each case on its own or in combination:

A preferred cycloalkyl moiety is cyclopentyl being optionally substituted by one or more nitro, cyano, C₁-C₆-alkyl, C₁-C₆-alkoxy groups.

A preferred heteroaryl moiety is pyridyl, pyrimidyl, pyrazolyl or thienyl.

Preference is given to compounds of formula I in which any alkyl part of the groups R¹ or R², which may be straight chained or branched, contains 1 to 9 carbon atoms, more preferably 2 to 6 carbon atoms, any alkenyl or alkynyl part of the substituents R¹ or R² contains 2 to 9 carbon atoms, more preferably 3 to 6 carbon atoms, any cycloalkyl part of the substituents R¹ or R² contains from 3 to 10 carbon atoms, preferably from 3 to 8 carbon atoms, more preferably from 3 to 6 carbon atoms, and any bicycloalkyl part of the substituents R¹ or R² contains from 7 to 9 carbon atoms. Any alkyl, alkenyl or alkynyl group may be linear or branched.

Compounds of formula I are preferred in which R¹ is not hydrogen.

Moreover, compounds of formula I are preferred in which R¹ represents a straight-chained or branched C₁-C₁₀-alkyl, in particular a branched C₃-C₁₀-alkyl group, a C₃-C₈-cycloalkyl, a C₅-C₉-bicycloalkyl, a C₃-C₈-cycloalkyl-C₁-C₆-alkyl, C₁-C₁₀-alkoxy-C₁-C₆-alkyl, or a phenyl group being optionally substituted by one to three C₁-C₁₀-alkyl or C₁-C₁₀-alkoxy groups.

Particular preference is given to compounds I in which R² represents a hydrogen atom or a C₁-C₁₀-alkyl group, in particular a hydrogen atom.

Moreover, particular preference is given to compounds I in which R² is methyl or ethyl.

If R¹ denotes an optionally substituted C₃-C₈-cycloalkyl group, preferably a cyclopentyl or cyclohexyl group, R² preferably represents a hydrogen atom or C₁-C₆-alkyl group.

Moreover, particular preference is given to compounds I in which R¹ and R² together with the interjacent nitrogen atom form an optionally substituted heterocyclic ring, preferably an optionally substituted C₃-C₇-heterocyclic ring, in particular a pyrrolidine, piperidine, tetrahydropyridine, in particular 1,2,3,6-tetrahydropyridine or azepane ring which is optionally substituted by one or more C₁-C₁₀-alkyl groups.

Besides, particular preference is given to compounds I in which X is chloro or bromo, especially chloro.

Moreover, preference is given to compounds I in which X is cyano or methoxy.

Furthermore, particular preference is given to compounds I in which X is ethoxy, n-propoxy, iso-propoxy, allyloxy, or 3-methylallyloxy.

Included in the scope of the present invention are (R) and (S) isomers of compounds of general formula I having a chiral center and the racemates thereof, and salts, N-oxides and acid addition compounds.

With respect to their use, particular preference is given to the compounds I compiled in the tables below. The groups mentioned in the tables for a substituent are furthermore for their part, independently of the combination in which they are mentioned, a particularly preferred embodiment of the respective substituents.

Table 1

Compounds of the formula I, in which X is chloro and R¹ and R² correspond to one row in Table A

Table 2

Compounds of the formula I, in which X is bromo and R¹ and R² correspond to one row in Table A

Table 3

Compounds of the formula I, in which X is cyano and R¹ and R² correspond to one row in Table A

Table 4

Compounds of the formula I, in which X is methoxy and R¹ and R² correspond to one row in Table A

Table 5

Compounds of the formula I, in which X is ethoxy and R¹ and R² correspond to one row in Table A

Table 6

Compounds of the formula I, in which X is n-propoxy and R¹ and R² correspond to one row in Table A

Table 7

Compounds of the formula I, in which X is iso-propoxy and R¹ and R² correspond to one row in Table A

Table 8

Compounds of the formula I, in which X is allyloxy and R¹ and R² correspond to one row in Table A

Table 9

Compounds of the formula I, in which X is 3-methylallyloxy and R¹ and R² correspond to one-row in Table A

TABLE A

| | |
|---|---|
| | I |
| 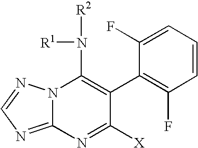 | |

| No. | R¹ | R² |
|---|---|---|
| A-1 | H | H |
| A-2 | CH₂CH₃ | H |
| A-3 | CH₂CH₃ | CH₃ |
| A-4 | CH₂CH₃ | CH₂CH₃ |
| A-5 | CH₂CH₂CH₃ | H |
| A-6 | CH₂CH₂CH₃ | CH₃ |
| A-7 | CH₂CH₂CH₃ | CH₂CH₃ |
| A-8 | CH₂CH₂CH₃ | CH₂CH₂CH₃ |
| A-9 | CH(CH₃)₂ | H |
| A-10 | CH(CH₃)₂ | CH₃ |
| A-11 | CH(CH₃)₂ | CH₂CH₃ |
| A-12 | (±) CH(CH₃)—CH₂CH₃ | H |
| A-13 | (±) CH(CH₃)—CH₂CH₃ | CH₃ |
| A-14 | (±) CH(CH₃)—CH₂CH₃ | CH₂CH₃ |
| A-15 | (S) CH(CH₃)—CH₂CH₃ | H |
| A-16 | (S) CH(CH₃)—CH₂CH₃ | CH₃ |
| A-17 | (S) CH(CH₃)—CH₂CH₃ | CH₂CH₃ |
| A-18 | (R) CH(CH₃)—CH₂CH₃ | H |
| A-19 | (R) CH(CH₃)—CH₂CH₃ | CH₃ |
| A-20 | (R) CH(CH₃)—CH₂CH₃ | CH₂CH₃ |
| A-21 | (±) CH(CH₃)—CH(CH₃)₂ | H |
| A-22 | (±) CH(CH₃)—CH(CH₃)₂ | CH₃ |
| A-23 | (±) CH(CH₃)—CH(CH₃)₂ | CH₂CH₃ |
| A-24 | (S) CH(CH₃)—CH(CH₃)₂ | H |
| A-25 | (S) CH(CH₃)—CH(CH₃)₂ | CH₃ |
| A-26 | (S) CH(CH₃)—CH(CH₃)₂ | CH₂CH₃ |
| A-27 | (R) CH(CH₃)—CH(CH₃)₂ | H |
| A-28 | (R) CH(CH₃)—CH(CH₃)₂ | CH₃ |
| A-29 | (R) CH(CH₃)—CH(CH₃)₂ | CH₂CH₃ |
| A-30 | (±) CH(CH₃)—C(CH₃)₃ | H |
| A-31 | (±) CH(CH₃)—C(CH₃)₃ | CH₃ |
| A-32 | (±) CH(CH₃)—C(CH₃)₃ | CH₂CH₃ |
| A-33 | (S) CH(CH₃)—C(CH₃)₃ | H |
| A-34 | (S) CH(CH₃)—C(CH₃)₃ | CH₃ |
| A-35 | (S) CH(CH₃)—C(CH₃)₃ | CH₂CH₃ |
| A-36 | (R) CH(CH₃)—C(CH₃)₃ | H |
| A-37 | (R) CH(CH₃)—C(CH₃)₃ | CH₃ |
| A-38 | (R) CH(CH₃)—C(CH₃)₃ | CH₂CH₃ |
| A-39 | CH₂C(CH₃)═CH₂ | H |
| A-40 | CH₂C(CH₃)═CH₂ | CH₃ |
| A-41 | CH₂C(CH₃)═CH₂ | CH₂CH₃ |
| A-42 | cyclopentyl | H |
| A-43 | cyclopentyl | CH₃ |
| A-44 | cyclopentyl | CH₂CH₃ |

| | |
|---|---|
| A-45 | —(CH₂)₂CH(CH₃)(CH₂)₂— |

The compounds I are suitable as fungicides. They have outstanding activity against a broad spectrum of phytopathogenic fungi, in particular from the classes of the Ascomycetes, Deuteromycetes, Phycomycetes and Basidiomycetes. Some of them act systemically, and they can be employed in crop protection as foliar- and soilacting fungicides.

They are especially important for controlling a large number of fungi on a variety of crop plants such as wheat, rye, barley, oats, rice, maize, grass, bananas, cotton, soya, coffee, sugar cane, grapevines, fruit species, ornamentals and vegetables such as cucumbers, beans, tomatoes, potatoes and cucurbits, and on the seeds of these plants.

Specifically, they are suitable for controlling the following plant diseases:

Alternaria species, Podosphaera species, Sclerotinia species, Physalospora canker on vegetables and fruit,

Botrytis cinerea (gray mold) an strawberries, vegetables, ornamentals and grapevines,

Corynespora cassiicola on cucumbers,

Colletotrichum species on fruit and vegetables,

Diplocarpon rosae on roses,

Elsinoe fawcetti and Diaporthe citri on citrus fruit,

Sphaerotheca species on cucurbits, strawberries and roses,

Cercospora species on peanuts, sugar beets and aubergines,

Erysiphe cichoracearum on cucurbits,

Leveillula taurica on paprika, tomatoes and aubergines,

Mycosphaerella species on apples and japanese apricot,

Phyllactinia kakicola, Gloesporium kaki on japanese apricot,

Gymnosporangium yamadae, Leptothyrium pomi, Podosphaera leucotricha and Gloedes pomigena on apples,

Cladosporium carpophilum on pears and japanese apricot,

Phomopsis species on pears,

Phytophthora species on citrus fruit, potatoes, onions, especially Phytophthora infestans on potatoes and tomatoes,

Blumeria graminis (powdery mildew) on cereals,

Fusariun- and Verticillium species on various plants,

Glomerella cingulata on tee,

Drechslera- and Bipolaris species on cereals and rice,

Mycosphaerella species on bananas and peanuts,

Plasmopara viticola on grapevines,

Personospora species on onions, spinach and chrysantemum,

Phaeoisariopsis vitis and Sphaceloma ampelina on grapefruits,

Pseudocercosporella herpotrichoides on wheat and barley,

Pseudoperonospora species on hop and cucumbers,

Puccinia species and Typhula species on cereals and turf,

Pyricularia oryzae on rice,

Rhizoctonia species on cotton, rice and turf,

Stagonospora nodoyum and Septoria triticb on wheat,

Uncinula necator on grapevines,

Ustilago species on cereals and sugar cane, and

Venturia species (scab) on apples and pears.

Moreover, the compounds I are suitable for controlling harmful fungi such as Paecilomyces variotii in the protection of materials (e.g. wood, paper, paint dispersions, fibers and tissues) and in the protection of stored products.

The compounds I are applied by treating the fungi, or the plants, seeds, materials or the soil to be protected against fungal infection, with a fungicidally active amount of the active ingredients. Application can be effected both before and after infection of the materials, plants or seeds by the fungi.

In general, the fungicidal compositions comprise from 0.1 to 95, preferably 0.5 to 90, % by weight of active ingredient. When used in crop protection, the rates of application are from 0.01 to 2.0 kg of active ingredient per ha, depending on the nature of the effect desired.

In the treatment of seed, amounts of active ingredient of from 0.001 to 0.1 g, preferably 0.01 to 0.05 g, are generally required per kilogram of seed.

When used in the protection of materials or stored products, the rate of application of active ingredient depends on the nature of the field of application and on the effect desired. Rates of application conventionally used in the protection of materials are, for example, from 0.001 g to 2 kg, preferably 0.005 g to 1 kg, of active ingredient per cubic meter of material treated.

The compounds I can be converted into the customary formulations, e.g. solutions, emulsions, suspensions, dusts, powders, pastes and granules. The use form depends on the particular purpose; in any case, it should guarantee a fine and uniform distribution of the compound according to the invention.

The formulations are prepared in a known manner, e.g. by extending the active ingredient with solvents and/or carriers, if desired using emulsifiers and dispersants, it also being possible to use other organic solvents as auxiliary solvents if water is used as the diluent. Auxiliaries which are suitable are essentially: solvents such as aromatics (e.g. xylene), chlorinated aromatics (e.g. chlorobenzenes), paraffins (e.g. mineral oil fractions), alcohols (e.g. methanol, butanol), ketones (e.g. cyclohexanone), amines (e.g. ethanolamine, dimethylformamide) and water; carriers such as ground natural minerals (e.g. kaolins, clays, talc, chalk) and ground synthetic minerals (e.g. highlydisperse silica, silicates); emulsifiers such as non-ionic and anionic emulsifiers (e.g. polyoxyethylene fatty alcohol ethers, alkylsulfonates and arylsulfonates) and dispersants such as lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates and fatty acids and their alkali metal and alkaline earth metal salts, salts of sulfated fatty alcohol glycol ether, condensates of sulfonated naphthalene and naphthalene derivatives with formaldehyde, condensates of naphthalene or of napthalenesulfonic acid with phenol or formaldehyde, polyoxyethylene octylphenyl ether, ethoxylated isooctylphenol, octylphenol, nonylphenol, alkylphenol polyglycol ethers, tributylphenyl polyglycol ethers, alkylaryl polyether alcohols, isotridecyl alcohol, fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, e.g. benzene, toluene, xylene, paraffin, tetrahydronaphthalene, alkylated naphthalenes or their derivatives, methanol, ethanol, propanol, butanol, chloroform, carbon tetrachloride, cyclohexanol, cyclohexanone, chlorobenzene, isophorone, strongly polar solvents, e.g. dimethylformamide, dimethyl sulfoxide, N-methylpyrrolidone and water.

Powders, materials for scattering and dusts can be prepared by mixing or concomitantly grinding the active substances with a solid carrier.

Granules, e.g. coated granules, impregnated granules and homogeneous granules, can be prepared by binding the active ingredients to solid carriers. Examples of solid carriers are mineral earths, such as silicas, silica gels, silicates, talc, kaolin, attaclay, limestone, lime, chalk, bole, leoss, clay, dolomite, diatomaceous earth, calcium sulfate, magnesium sulfate, magnesium oxide, ground synthetic materials, fertilizers, e.g. ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise of from 0.01 to 95% by weight, preferably from 0.1 to 90% by weight, of the active ingredient. The active ingredients are employed in a purity of from 90% to 100%., preferably 95% to 100% (according to NMR spectrum).

The following are exemplary formulations:

I. 5 parts by weight of a compound according to the invention are mixed intimately with 95 parts by weight of finely divided kaolin. This gives a dust which comprises 5% by weight of the active ingredient.

II. 30 parts by weight of a compound according to the invention are mixed intimately with a mixture of 92 parts by weight of pulverulent silica gel and 8 parts by weight of paraffin oil which had been sprayed onto the surface of this silica gel. This gives a formulation of the active ingredient with good adhesion properties (comprises 23% by weight of active ingredient).

III. 10 parts by weight of a compound according to the invention are dissolved in a mixture composed of 90 parts by weight of xylene, 6 parts by weight of the adduct of 8 to 10 mol of ethylene oxide and 1 mol of oleic acid N-monoethanolamide, 2 parts by weight of calcium dodecylbenzenesulfonate and 2 parts by weight of the adduct of 40 mol of ethylene oxide and 1 mol of castor oil (comprises 9% by weight of active ingredient).

IV. 20 parts by weight of a compound according to the invention are dissolved in a mixture composed of 60 parts by weight of cyclohexanone, 30 parts by weight of isobutanol, 5 parts by weight of the adduct of 7 mol of ethylene oxide and 1 mol of isooctylphenol and 5 parts by weight of the adduct of 40 mol of ethylene oxide and 1 mol of castor oil (comprises 16% by weight of active ingredient).

V. 80 parts by weight of a compound according to the invention are mixed thoroughly with 3 parts by weight of sodium diisobutylnaphthalene-alpha-sulfonate, 10 parts by weight of the sodium salt of a lignosulfonic-acid from a sulfite waste liquor and 7 parts by weight of pulverulent silica gel, and the mixture is ground in a hammer mill (comprises 80% by weight of active ingredient).

VI. 90 parts by weight of a compound according to the invention are mixed with 10 parts by weight of N-methyl-α-pyrrolidone, which gives a solution which is suitable for use in the form of microdrops (comprises 90% by weight of active ingredient).

VII. 20 parts by weight of a compound according to the invention are dissolved in a mixture composed of 40 parts by weight of cyclohexanone, 30 parts by weight of isobutanol, 20 parts by weight of the adduct of 7 mol of ethylene oxide and 1 mol of isooctylphenol and 10 parts by weight of the adduct of 40 mol of ethylene oxide and 1 mol of castor oil. Pouring the solution into 100,000 parts by weight of water and finely distributing it therein gives an aqueous dispersion which comprises 0.02% by weight of the active ingredient.

VIII. 20 parts by weight of a compound according to the invention are mixed thoroughly with 3 parts by weight of sodium diisobutylnaphthalene-α-sulfonate, 17 parts by weight of the sodium salt of a lignosulfonic acid from a sulfite waste liquor and 60 parts by weight of pulverulent silica gel, and the mixture is ground in a hammer mill. Finely distributing the mixture in 20,000 parts by weight of water gives a spray mixture which comprises 0.1% by weight of the active ingredient.

The active ingredients can be used as such., in the form of their formulations or the use forms prepared therefrom, e.g. in the form of directly sprayable solutions, powders, suspensions or dispersions, emulsions, oil dispersions, pastes, dusts, materials for spreading, or granules, by means of spraying, atomizing, dusting, scattering or pouring. The use forms depend entirely on the intended purposes; in any case, this is intended to guarantee the finest possible distribution of the active ingredients according to the invention. Aqueous use forms can be prepared from emulsion concentrates, pastes or wettable powders (sprayable powders, oil dispersions) by adding water. To prepare emulsions, pastes or oil dispersions, the substances as such or dissolved in an oil or solvent, can be homogenized in water by means of wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within substantial ranges. In general, they are from 0.0001 to 10%, preferably from 0.01 to 1%.

The active ingredients may also be used successfully in the ultra-low-volume process (ULV), it being possible to apply formulations comprising over 95% by weight of active ingredient, or even the active ingredient without additives.

Various types of oils, herbicides, fungicides, other pesticides, or bactericides may be added to the active ingredients, if appropriate also only immediately prior to use (tank mix). These agents can be admixed with the agents according to the invention in a weight ratio of 1:10 to 10:1.

In the use form as fungicides, the compositions according to the invention can also be present together with other active ingredients, e.g. with herbicides, insecticides, growth regulators, fungicides or else with fertilizers. Mixing the compounds I or the compositions comprising them in the use form as fungicides with other fungicides frequently results in a broader fungicidal spectrum of action.

The following list of fungicides, together with which the compounds according to the invention can be used, is intended to illustrate the possible combinations, but not to impose any limitation:

sulfur, dithiocarbamates and their derivatives, such as iron(III) dimethyldithiocarbamate, zinc dimethyldithiocarbamate, zinc ethylenebisdithiocarbamate, manganese ethylenebis, dithiocarbamate, manganese zinc ethylenediaminebisdithiocarbamate, tetramethylthiuram disulfide, ammonia complex of zinc (N,N-ethylenebisdithiocarbamate), ammonia complex of zinc (N,N′-propylenebisdithiocarbamate), zinc (N,N′-propylenebisdithiocarbamate), N,N′-polypropylenebis(thiocarbamoyl)disulfide;

nitro derivatives, such as dinitro(1-methylheptyl)phenyl crotonate, 2-sec-butyl-4,6-dinitrophenyl 3,3-dimethylacrylate, 2-sec-butyl-4,6-dinitrophenylisopropyl carbonate, diisopropyl 5-nitro-isophthalate;

heterocyclic substances, such as 2-heptadecyl-2-imidazoline acetate, 2,4-dichloro-6-(o-chloroanilino)-s-triazine, O,O-diethyl phthalimidophosphonothioate, 5-amino-1-[bis(dimethylamino)phosphinyl]-3-phenyl-1,2,4-triazole, 2,3-dicyano-1,4-dithioanthraquinone, 2-thio-1,3-dithiolo[4,5-b]quinoxaline, methyl 1-(butylcarbamoyl)-2-benzimidazolecarbamate, 2-methoxycarbonylaminobenzimidazole, 2-(2-furyl)benzimidazole, 2-(4-thiazolyl)benzimidazole, N-(1,1,2,2-tetrachloroethylthio)tetrahydrophthalimide, N-trichloromethylthiotetrahydrophthalimide, N-trichloromethylthiophthalimide, 5-Chloro-2-cyano-4-p-tolyl-imidazole-1-sulfonic acid dimethylamide, N-dichlorofluoromethylthio-N′,N′-dimethyl-N-phenylsulfo-diamide, 5-ethoxy-3-trichloromethyl-1,2,3-thiadiazole, 2-thiocyanatomethylthiobenzothiazole, 1,4-dichloro-2,5-dimethoxybenzene, 4-(2-chlorophenylhydrazono)-3-methyl-5-isoxazolone, pyridine-2-thiol 1-oxide, 8-hydroxyquinoline or its copper salt, 2,3-dihydro-5-carboxanilido-6-methyl-1,4-oxathiine, 2,3-dihydro-5-carboxanilido-6-methyl-1,4-oxathiine 4,4-dioxide, 2-methyl-5,6-dihydro-4H-pyran-3-carboxanilide, 2-methylfuran-3-carboxanilide, 2,5-dimethylfuran-3-carboxanilide, 2-Chloro-N-(4′-chloro-biphenyl-2-yl)-nicotinamide, 2,4,5-trimethylfuran-3-carboxanilide, N-cyclohexyl-2,5-dimethylfuran-3-carboxamide, N-cyclohexyl-N-methoxy-2,5-dimethylfuran-3-carboxamide, 2-methylbenzanilide, 2-iodobenzanilide, N-formyl-N-morpholine-2,2,2-trichloroethyl acetal, piperazine-1,4-diylbis-1-(2,2,2-trichloroethyl)formamide, 1-(3,4-dichloroanilino)-1-formylamino-2,2,2-trichloroethane; 2,6-dimethyl-N-tridecylmorpholine or its salts, 2,6-dimethyl-N-cyclododecylmorpholine or its salts, N-[3-(p-tert-butylphenyl)-2-methylpropyl]-cis-2,6-dimethyl-morpholine, N-[3-(p-tert-butylphenyl)-2-methylpropyl]-piperidine, 1-[2-(2,4-dichlorophenyl)-4-ethyl-1,3-dioxolan-2-yl-ethyl]-1H-1,2,4-triazole, 1-[2-(2,4-dichlorophenyl)-4-n-propyl-1,3-dioxolan-2-yl-ethyl]-1H-1,2,4-triazole, N-(n-propyl)-N-(2,4,6-trichlorophenoxyethyl)-N′-imidazolyl-urea, 1-(4-chlorophenoxy)-3,3-dimethyl-1-(1H-1,2,4-triazol-1-yl)-2-butanone, 1-(4-chlorophenoxy)-3,3-dimethyl-1-(1H-1,2,4-triazol-1-yl)-2-butanol, (2RS,3RS)-1-[3-(2-chlorophenyl)-2-(4-fluorophenyl)-oxiran-2-ylmethyl]-1H-1,2,4-triazole, α-(2-chlorophenyl)-α-(4-chlorophenyl)-5-pyrimidinemethanol, 5-butyl-2-dimethylamino-4-hydroxy-6-methylpyrimidine, bis(p-chlorophenyl)-3-pyridinemethanol, 1,2-bis(3-ethoxycarbonyl-2-thioureido)benzene, 1,2-bis(3-methoxycarbonyl-2-thioureido)benzene,

strobilurines such as azoxystrobin, kresoxim methyl, methyl-E-methoxyimino-[α-(2-phenoxyphenyl)]-acetamide, methyl E-methoxyimino-[α-(2,5-dimethylphenoxy)-o-tolyl]acetamide, picoxystrobin, pyraclostrobin, trifloxystrobin, anilinopyrimidines such as N-(4,6-dimethylpyrimidin-2-yl)aniline, N-[4-methyl-6-(1-propynyl)pyrimidin-2-yl]-aniline, N-[4-methyl-6-cyclopropylpyrimidin-2-yl]aniline, phenylpyrroles such as 4-(2,2-difluoro-1,3-benzodioxol-4-yl)pyrrole-3-carbonitrile,

cinnamamides such as 3-(4-chlorophenyl)-3-(3,4-dimethoxyphenyl)acryloylmorpholine, 3-(4-fluorophenyl)-3-(3,4-dimethoxy-phenyl)acryloylmorpholine,

and a variety of fungicides such as dodecylguanidine acetate, 3-[3-(3,5-dimethyl-2-oxycyclohexyl)-2-hydroxyethyl]glutarimide, hexachlorobenzene, methyl N-(2,6-dimethylphenyl)-N-(2-furoyl)-DL-alaninate, DL-N-(2,6-dimethylphenyl)-N-(2′-methoxyacetyl)-alanine methyl ester, N-(2,6-dimethylphenyl)-N-chloroacetyl-D,L-2-amino-butyrolactone, DL-N-(2,6-dimethylphenyl)-N-(phenylacetyl)alanine methyl ester, 5-methyl-5-vinyl-3-(3,5-dichlorophenyl)-2,4-dioxo-1,3-oxazolidine, 3-[3,5-dichlorophenyl(5-methyl-5-methoxymethyl]-1,3-oxazolidine-2,4-dione, 3-(3,5-dichlorophenyl)-1-isopropylcarbamoylhydantoin, N-(3,5-dichlorophenyl)-1,2-dimethylcyclopropane-1,2-dicarbowximide, 2-cyano-[N-(ethylaminocarbonyl)-2-methoximino]acetamide, 3,5-Dichloro-N-(3-chloro-1-ethyl-1-methyl-2-oxo-propyl)-4-methyl-benzamide, 1-(3-Bromo-6-methoxy-2-methyl-phenyl)-1-(2,3,4-trimethoxy-6-methyl-phenyl)-methanone, 1-[2-(2,4-dichloro-phenyl)pentyl]-1H-1,2,4-triazole, 2,4-difluoro-α-(1H-1,2,4-triazolyl-1-methyl)benzhydryl alcohol, N-(3-chloro-2,6-dinitro-4-trifluoromethylphenyl)-5-trifluoromethyl-3-chloro-2-aminopyridine, 1-((bis(4-fluorophenyl)methylsilyl)methyl)-1H-1,2,4-triazole.

### SYNTHESIS EXAMPLES

With due modification of the starting compounds, the protocol shown in the synthesis example below was used for obtaining further compounds I. The resulting compounds I, together with physical data, are listed in the Table I which follows.

### Example 1

### Preparation of 5,7-dihydroxy-6-(2,6-difluorophenyl)-[1,2,4]-triazolo[1,5-α]-pyrimidine

A mixture of 3-amino-1,2,4-triazole (14 g), diethyl, (2,6-difluorophenyl)-malonate (0, 17 mol, cf. EP-A 10 02 788) and tributy-, amine (50 ml) was heated at about 180° C. for six hours. The reaction mixture was cooled to about 70° C. 200 ml of 10% aqueous sodium hydroxide solution were added and the reaction mixture were stirred for 30 minutes. The phases were separated, the aqueous phase was extracted with diethyl ether. The aqueous phase is acidified. The precipitate was filtered off and dried to yield 40 g of the title compound.

### Example 2

### Preparation of 5,7-dichloro-6-(2,6-difluorophenyl)-[1,2,4]-triazolo[1,5-α]-pyrimidine

A mixture of 5,7-dihydroxy-6-(2,6-difluorophenyl)-[1,2,4]-triazolo-[1,5-α]pyrimidine (30 g, obtained from Ex. 1) and phosphorous oxychloride-(50 ml) was refluxed for about eight hours. Phosphorous oxychloride was partly-distilled off. The residue was poured into a mixture of dichloromethane and water. The organic layer was separated, dried and filtered. The filtrate was concentrated in vacuo; to yield 28 g of the title compound of mp. 121° C.

### Example 3

### Preparation of 5-chloro-6-(2,6-difluorophenyl)-7-isopropylamino-[1,2,4]-triazolo[1,5-α]pyrimidine [1-3]

A mixture of isopropylamine (1,5 mmol), triethylamine (1,5 mmol) and dichloromethane (10 ml) was added to a mixture of 5,7-dichloro-6-(2,6-difluorophenyl)-[1,2,4]-triazolo[1,5-α]-pyrimidine (1,5 mmol, obtained from Ex. 2) and dichloromethane (20 ml) under stirring. The reaction mixture was stirred for about 16 hours at about 20 to 25° C. and subsequently washed with dilute hydrocloric acid (5%). The organic layer was separated, dried and filtered. The filtrate was evaporated under reduced pressure and the residue was chromatographed to yield 0,43 g of the title compound of mp. 169° C.

### Example 4

### Preparation of 5-cyano-6-(2,6-difluorophenyl)-7-(4-methylpiperidin-1-yl)-[1,2,4]-triazolo[1,5-α]pyrimidine [I-18]

A mixture of 5-chloro-6-(2,6-difluorophenyl)-7-(4-methylpiperidin-1-yl)-[1,2,4]-triazolo-[1,5-α]-pyrimidine (0, 1 mol; I-4) and tetraethylammonium cyanide (0,25 mol) in 750 ml Dimethylformamide (DMF) (750 ml) was stirred for 16 hours at about 20 to 25° C. To this mixture water was added and methyl tert.butyl ether (MTBE), the organic phase was separated, washed with water, dried and filtered. The filtrate was evaporated and the residue was chromatographed to yield 6,95 g of the title compound of mp. 212° C.

### Example 5

### Preparation of 5-methoxy-6-(2,6-difluorophenyl)-7-(diethylamino)-[1,2,4]-triazolo[1,5-α]pyrimidine [I-19]

To a solution of 5-chloro-6-(2,6-difluorophenyl)-7-(diethylamino)-[1,2,4]-triazolo-[1,5-α]-pyrimidine (65 mmol; I-6) in 400 ml dry methanol was added a 30% solution of sodium methanolate (71,5 mmol) at about 20 to 25° C. This mixture was further stirred for 16 hours. Methanol was evaporated and the residue was dissolved with dichloromethane. The organic phase was washed with water, dried and filtered. The filtrate was evaporated and the residue was chromatographed to yield 16,3 g of the title compound of mp. 153° C.

TABLE I

| | |
|---|---|
| | I |
| 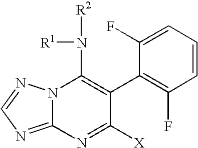 | |

| | | | | phys. data |
|---|---|---|---|---|
| No. | R¹ | R² | X | (m.p. [° C.]) |
| I-1 | H | H | Cl | 250 |
| I-2 | CH₂C(CH₃)═CH₂ | CH₂CH₃ | Cl | 96 |
| I-3 | CH(CH₃)₂ | H | Cl | 169 |
| I-4 | —(CH₂)₂CH(CH₃)(CH₂)₂— | | Cl | 196 |
| I-5 | cyclopentyl | H | Cl | 165 |
| I-6 | CH₂CH₃ | CH₂CH₃ | Cl | 159 |
| I-6 | CH₂CH₃ | CH₂CH₃ | Cl | 159 |
| I-7 | CH₂CH₂CH₃ | CH₂CH₂CH₃ | Cl | 125 |
| I-8 | CH(CH₃)₂ | CH₃ | Cl | 168 |
| I-9 | (±) CH(CH₃)—CH₂CH₃ | H | Cl | 184 |
| I-10 | (S) CH(CH₃)—CH₂CH₃ | H | Cl | 176 |
| I-11 | (R) CH(CH₃)—CH₂CH₃ | H | Cl | 176 |
| I-12 | (±) CH(CH₃)—CH(CH₃)₂ | H | Cl | 157 |
| I-13 | (S) CH(CH₃)—CH(CH₃)₂ | H | Cl | 149 |
| I-14 | (R) CH(CH₃)—CH(CH₃)₂ | H | Cl | 149 |
| I-15 | (±) CH(CH₃)—C(CH₃)₃ | H | Cl | 160 |
| I-16 | (S) CH(CH₃)—C(CH₃)₃ | H | Cl | 175 |
| I-17 | (R) CH(CH₃)—C(CH₃)₃ | H | Cl | 175 |
| I-18 | —(CH₂)₂CH(CH₃)(CH₂)₂— | | CN | 212 |
| I-19 | CH₂CH₃ | CH₂CH₃ | OCH₃ | 153 |

Examples of the action against harmful fungi

The fungicidal action of the compounds of the formula I was demonstrated by the following experiments:

The active compounds, separately or together, were formulated as a 10% emulsion in a mixture of 70% by weight of cyclohexanone, 20% by weight of Nekanil® LN (Lutensol® AP6, wetting agent having emulsifying and dispersant action based on ethoxylated alkylphenols) and 10% by weight of Wettol® EM (nonionic emulsifier based on ethoxylated castor oil) and diluted with water to the desired concentration.

Biological activity trial—Fungicidal control of early blight on tomatoes (Alternaria solani)

Young seedlings of tomato plants of the variety “Groβe Fleischtomate St. Pierre” were grown in pots to the 2 to 4 leaf stage. These plants were sprayed to run-off with an aqueous suspension, containing the concentration of active ingredient mentioned in the table below, prepared from a stock solution containing 10% of the active ingredient, 85% cyclohexanone and 5% emulsifier. The next day, the treated plants were inoculated with an aqueous suspension of Alternaria solani containing 0,2×10⁶ spores per ml. Then the trial plants were immediately transferred to a humid chamber. After 6 days at 20 to 23° C. and a relative humidity close to 100%, the extent of fungal attack on the leaves was visually assessed as % diseased leaf area. In this test, the plants which had been treated with 250 ppm of compounds I-2 to I-9, I-11, I-12, I-14, I-15 and I-17 showed an infection of not more than 1%, whereas the untreated plants were infected to 90%.

Active compounds A and B known from WO-A 98/46608 were used as comparison compounds:

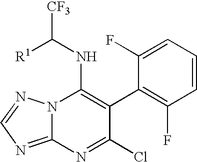

| No. | known from | R¹ |
|---|---|---|
| A | WO-A 98/46608, No. 5 | hydrogen |
| B | WO-A 98/46608, No. 7 | methyl |

Comparison trial—Control of gray mould (Botrytis cinerea) on fruit slices of green pepper

Fruit slices of green pepper were sprayed to run-off with an aqueous suspension, containing the concentration of active ingredient or their mixture mentioned in the table below, prepared from a stock solution containing 10% of the active ingredient; 85% cyclohexanone and 5% emulsifier. After 2 hours the sprayed-on layer had dried, the disks were inoculated with a spore suspension of Botrytis cinerea containing 1,7×10⁶ spores per ml in 2 wt. % aqueous biomalt solution. The infected fruit slices were then incubated in chambers with high humidity for four days at 18-20° C. The fruit slice area under fungal attack was then assessed visually in percent.

In this test, the plants which had been treated with 500 ppm of compounds I-2, I-8 and I-14, resp., showed an infection of not more than 3%, whereas the the plants treated with 500 ppm of comparison compounds A and B, resp., were infected to 100 and 20%, and the unteated plants were infected to 100%.

1. 6-(2,6-Difluoro-phenyl)-triazolopyrimidines of formula I 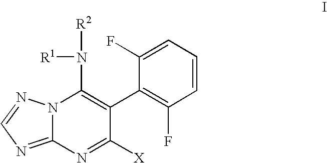
in which
R¹ and R² independently denote hydrogen or
C₁-C₁₀-alkyl, C₂-C₁₀-alkenyl, C₂-C₁₀-alkynyl, or C₄-C₁₀-alkadienyl,
C₃-C₁₀-cycloalkyl, phenyl, naphthyl, or
5- or 6-membered heterocyclyl, containing one to four nitrogen atoms or one to three nitrogen atoms and one sulfur or oxygen atom, or
5- or 6-membered heteroaryl, containing one to four nitrogen atoms or one to three nitrogen atoms and one sulfur or oxygen atom, or
where R¹ and R² radicals may be unsubstituted or may carry one to three groups Rₐ,
R^{a} is cyano, nitro, hydroxyl, C₁-C₆-alkyl, C₁-C₆-haloalkyl, C₃-C₆-cycloalkyl, C₁-C₆-alkoxy, C₁-C₆-haloalkoxy, C₁-C₆-alkylthio, C₁-C₆-alkylamino, di-C₁-C₆-alkylamino, C₂-C₆-alkenyl, C₂-C₆-alkenyloxy, C₂-C₆-alkynyl, C₃-C₆-alkynyloxy and C₁-C₄-alkylenedioxy; or
R¹ and R² together with the interjacent nitrogen atom represent a 5- or 6-membered heterocyclic ring, containing one to four nitrogen atoms or one to three nitrogen atoms and one sulfur or oxygen atom, which may be substituted by one to three R^{a} radicals;
x is halogen, cyano, C₁-C₆-alkoxy, C₁-C₆-haloalkoxy or C₃-C₈-alkenyloxy.

2. Compounds of formula I according to claim 1, in which
R¹ is straight chained or branched C₁-C₆-alkyl, C₂-C₆-alkenyl, or C₃-C₉-cycloalkyl, and
R² is hydrogen or C₁-C₆-alkyl, or
R¹ and R² together with the interiacent nitrogen atom represent a heterocyclic ring with 5 or 6 carbon atoms being optionally substituted with one or two C₁-C₆-alkyl groups.

3. Compounds according to claims 1 or 2 in which R² is hydrogen.

4. Compounds according to claims 1 to 3 in which X is halogen.

5. A process for the preparation of compounds of formula I as defined in claim 4 which comprises reacting 5,7-dihalogen-6-(2,6-difluoro-phenyl)-triazolopyrimidines of formula II 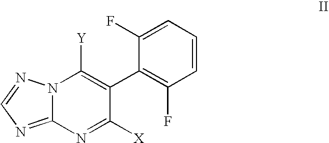
in which Y is halogen with an amine of formula III 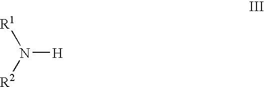
in which R¹ and R² are defined as for formula I to produce compounds of formula I.

6. A composition suitable for controlling phytopathogenic fungi, comprising a solid or liquid carrier and a compound of the formula I as claimed in claim 1.

7. A method for controlling phytopathogenic fungi, which comprises treating the fungi or the materials, plants, the soil or the seed to be protected against fungal attack with an effective amount of a compound of the formula I as claimed in claim 1.
